(19) United States
(12) Patent Application Publication
(10) Pub. No.: US 2024/0100302 A1
(43) Pub. Date: Mar. 28, 2024

(54) ULTRASOUND AIDED POSITIONING OF AN INTRAVENOUS CATHETER

(71) Applicant: Nexus Medical, LLC, Lenexa, KS (US)

(72) Inventors: W. Cary Dikeman, Leawood, KS (US); Andrew Rellihan, Leawood, KS (US)

(21) Appl. No.: 18/475,987

(22) Filed: Sep. 27, 2023

Related U.S. Application Data
(60) Provisional application No. 63/410,712, filed on Sep. 28, 2022.

(51) Int. Cl. A61M 25/01; A61M 5/42

(52) U.S. Cl. CPC A61M 25/0105; A61M 5/427; A61M 2025/0166; A61M 2205/3375; A61M 2205/502; A61M 2205/583

(57) ABSTRACT

Systems, methods, and devices for monitoring and facilitating insertion and positioning of an intravenous catheter within a vein of a patient. Real-time ultrasound data is used to monitor various parameters associated with the selected vein and catheter to aid in selection and positioning of the catheter during an insertion procedure.

### RELATED APPLICATIONS

This non-provisional patent application claims priority benefit, with regard to all common subject matter, of earlier-filed U.S. Provisional Patent Application No. 63/410,712, filed on Sep. 28, 2022, and entitled “ULTRASOUND AIDED POSITIONING OF AN INTRAVENOUS CATHETER.” The identified earlier-filed provisional patent application is hereby incorporated by reference in its entirety into the present application.

### BACKGROUND

### 1. Field

Embodiments of the present disclosure relate to intravenous catheters. More specifically, embodiments of the present disclosure relate to ultrasound aided positioning of an intravenous catheter.

### 2. Related Art

Intravenous catheters may be used to administer various medications into the blood stream of a patient. In particular, peripheral intravenous (IV) therapy is a common procedure performed in modern hospitals by medical personnel. However, various catheter parameters effect the blood flow velocity rate of the patient thereby complicating the selection of an appropriately sized catheter. Additionally, in many cases it is difficult for medical personnel to locate and select a suitable blood vessel for placement of the catheter. Poor insertion techniques and improper placement of an intravenous catheter may result in any of pre-mature catheter failure, medical complications, and treatment delays.

The positioning of the catheter within the vein of the patient also effects the blood flow velocity rate. As such, it may be difficult to achieve a proper hemodilution ratio of the patient for effective administration of the medication, which may lead to various complications. For example, a hemodilution ratio that is high leads to greater dilution of medication in the vascular system of the patient. Conversely, a hemodilution ratio that is too low may prevent proper integration or dilution of the medication into the patient's blood stream.

About one third of patients are recognized as having ‘difficult access’ in terms of intravenous insertion. Further, lack of necessary skill in medical personnel often results in multiple insertion attempts and training for proper insertion techniques is time consuming, expensive, and challenged by staff turnover.

### SUMMARY

Embodiments of the present disclosure solve the above-mentioned problems by providing a method and system for monitoring and positioning a catheter within a vein of a patient using ultrasound technology. Further still, embodiments of the present disclosure provide artificial intelligence for identifying anatomical structures within real-time ultrasound data, as well as training, guiding, and instructing medical personnel through an intravenous insertion procedure.

In some aspects, the techniques described herein relate to a method of inserting an intravenous catheter into a blood vessel of a patient, the method including receiving a first set of ultrasound data associated with the blood vessel diameter from an ultrasound device prior to an insertion of the intravenous catheter, estimating a first blood flow velocity rate associated with the blood vessel based on the first set of ultrasound data, receiving a second set of ultrasound data associated with the blood vessel depth from the ultrasound device before insertion of the intravenous catheter, identifying a plurality of positioning parameters of the intravenous catheter based at least in part on the second set of ultrasound data, the plurality of positioning parameters including an angle of insertion of the intravenous catheter and a depth of the vein.

In some aspects, the techniques described herein relate to a method, wherein the second set of ultrasound data includes a cross-section image or diameter of the blood vessel and the intravenous catheter, and a side image of the blood vessel and the intravenous catheter.

In some aspects, the techniques described herein relate to a method, further including automatically detecting one or more edges of the blood vessel within the first set of ultrasound data.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present disclosure will be apparent from the following detailed description of the embodiments and the accompanying drawing figures.

### BRIEF DESCRIPTION OF THE DRAWING FIGURES

Embodiments of the present disclosure are described in detail below with reference to the attached drawing figures, wherein:

FIG. 1 illustrates an exemplary system for intravenous insertion relating to some embodiments;

FIG. 2 illustrates an exemplary ultrasound relating to some embodiments;

FIG. 3 illustrates an exemplary operational environment of an insertion region relating to some embodiments;

FIG. 4A illustrates an exemplary user interface relating to some embodiments;

FIG. 4B illustrates an exemplary user interface relating to some embodiments;

FIG. 5A illustrates an exemplary user interface relating to some embodiments;

FIG. 5B illustrates an exemplary user interface relating to some embodiments;

FIG. 5C illustrates an exemplary user interface relating to some embodiments;

FIG. 6 illustrates an exemplary method of inserting an intravenous catheter into a blood vessel of a patient relating to some embodiments;

FIG. 7 illustrates an exemplary system diagram of a data system relating to some embodiments;

FIG. 8A illustrates an exemplary user interface relating to some embodiments;

FIG. 8B illustrates an exemplary user interface relating to some embodiments;

FIG. 9A illustrates an exemplary table showing catheter insertion records corresponding to a plurality of medical personnel relating to some embodiments;

FIG. 9B illustrates an exemplary user interface relating to some embodiments;

FIG. 9C illustrates an exemplary user interface including an insertion record relating to some embodiments;

FIG. 10 illustrates an exemplary method of providing a machine learning model relating to some embodiments; and

FIG. 11 illustrates an exemplary method of guiding intravenous catheter insertion relating to some embodiments.

The drawing figures do not limit the present disclosure to the specific embodiments disclosed and described herein. The drawings are not necessarily to scale, emphasis instead being placed upon clearly illustrating the principles of the present disclosure.

### DETAILED DESCRIPTION

The following detailed description references the accompanying drawings that illustrate specific embodiments in which the present disclosure can be practiced. The embodiments are intended to describe aspects of the present disclosure in sufficient detail to enable those skilled in the art to practice the present disclosure. Other embodiments can be utilized and changes can be made without departing from the scope of the present disclosure. The following detailed description is, therefore, not to be taken in a limiting sense. The scope of the present disclosure is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the technology can include a variety of combinations and/or integrations of the embodiments described herein.

Embodiments of the present disclosure provide improvements to intravenous catheter insertion by employing ultrasound technology. Real-time ultrasound data may be utilized to provide a visual display to medical personnel to aid in vein selection, catheter selection, catheter positioning, and other logistics and operational selections associated with intravenous catheter insertion. Further, embodiments are contemplated in which machine learning and other forms of artificial intelligence such as computer vision may be implemented to provide any of identification of anatomical structures, instruct medical personnel through an insertion procedure, select a suitable blood vessel and catheter, as well as other functions as will be described in further detail below.

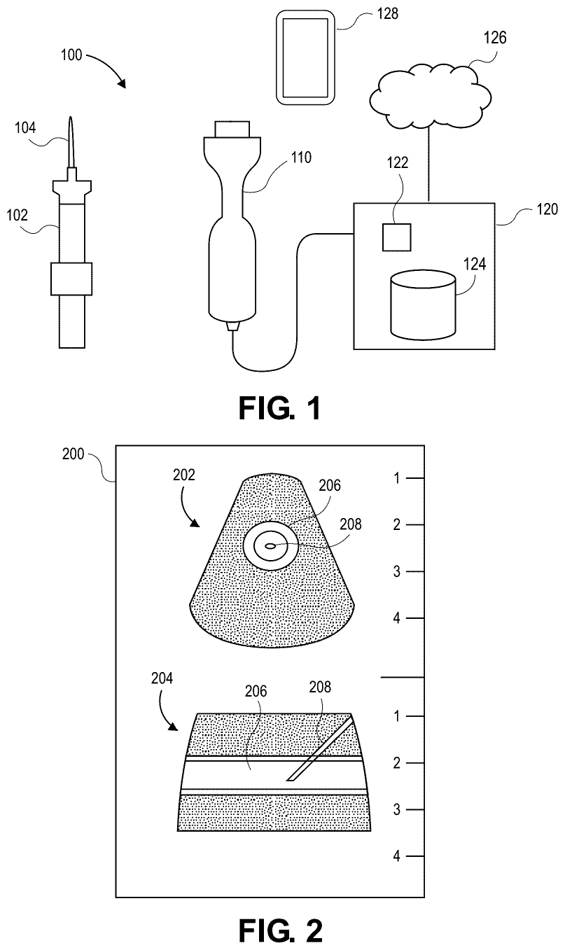

FIG. 1 illustrates one example of a system 100 for intravenous insertion relating to some embodiments. In some embodiments, the system 100 includes a catheter 102 including an insertion portion 104. In some such embodiments, the insertion portion 104 comprises a needle configured to be inserted into a patient's tissue. In some embodiments, the system 100 further comprises an ultrasound device 110. In some embodiments, the ultrasound device 110 is configured to transmit and monitor ultrasonic waves as part of a sonography procedure.

In some embodiments, the system 100 further comprises a device 120 coupled to the ultrasound device 110. In some embodiments, the device 120 comprises at least one processor such as processor 122, as shown, for processing ultrasound data collected by the ultrasound device 110. In some embodiments, the device 120 comprises a controller, computer, or other form of control unit or similar device. In some embodiments, the device 120 further comprises a data store 124 configured to store data associated with the device 120. For example, in some embodiments, the data store 124 stores ultrasound data captured by the ultrasound device 110.

In some embodiments, the device 120 may be communicatively coupled to a network 126 such as a wired or wireless network. For example, in some embodiments, the device 120 may be coupled to a cloud data store. Accordingly, the device 120 may be configured to transmit at least a portion of data collected by the ultrasound device 110 or other devices to the cloud data store via the network 126 such that the data may be stored remotely and/or transmitted elsewhere. Additionally, it should be understood that the communication connection with the network 126 may be bidirectionally, for example, such that data may be received by the device 120 from the network 126.

In some embodiments, a machine learning model may be included on the device 120, for example, the device 120 may include a machine learning algorithm stored on the data store 124. Said machine learning model may be trained based on historical ultrasound data and may be configured for any combination of identifying anatomical structures within real-time ultrasound data, instructing and/or guiding medical personnel through a catheter insertion operation, or grading medical personnel on insertion operation technique.

In some embodiments, a user device 128 may be included. The user device 128 may comprise any of a mobile device such as a smart phone, tablet, laptop computer, or another user device such as a desktop computer. In some embodiments, the user device 128 includes a processor, data store, and display configured to display a user interface. In some embodiments, a software application may be executed on the user device 128. For example, the user device 128 may store and execute an application for aiding in a catheter insertion procedure. In some embodiments, the user device 128 may be communicatively coupled to the device 120 via any of a wired connection or a wireless connection such as network 126.

In some embodiments, the user device 128 may be configured to automatically and/or manually record and store information associated with the catheter insertion procedure. For example, the user device 128 may be configured for hands free operation such that a medical personnel may operate the user device 128 and provide information to the user device 128 by uttering verbal phrases. As one example, the medical personnel may verbally read out outputs from the ultrasound device such as any of a vein diameter, hemodilution ratio, catheter to vein ratio, centimeter per second blood flow velocity, physician order for an infusion flow rate of medication to be delivered, distance from the skin surface to top of vein, angle of insertion, and IV catheter gauge, as well as other parameters associated with the catheter insertion procedure such that the user device 128 can record and store the parameters. Here, the user device 128 may be configured with voice recognition to identify verbally uttered phrases from the medical personnel and record ultrasound and other insertion data within a data store. Said handsfree operation to record parameters allows the medical personnel to continue the insertion procedure while maintaining aseptic no touch technique (ANTT) to thereby reduce touch contamination during the insertion procedure. Alternatively, or additionally, in some embodiments, one or more insertion parameters such as, number of insertion attempts, angle of insertion, diameter, depth, blood flow velocity, vein purchase, and final tip position may be automatically detected and recorded based on real-time ultrasound data.

In some embodiments, an ultrasound application may be included on the user device 128 to automatically receive and provide parameters and other information associated with the insertion procedure. For example, the user device 128 may receive a distance from the skin surface to the surface of the selected vein and use the distance value as an input to calculate a recommended catheter length based on a selected angle of insertion. In some embodiments, one or more trigonometric functions may be used to determine a recommended catheter length. Further, in some embodiments, the user device 128 may be configured to automatically select a particular catheter device from a catheter data store based on one or more input parameters.

Alternatively, or additionally, in some embodiments, the user device 128 may be configured to automatically update and store parameters based on a communication connection with the device 120 and ultrasound device 110. For example, a real-time image stream of ultrasound data may be received from the ultrasound device 110 and the user device 128 or device 120 may be configured to analyze the real-time ultrasound data to automatically determine one or more parameters. Said one or more parameters are then stored within a data store of the respective device and/or transmitted to the network 126 for remote cloud storage. Further still, it should be understood that action may be taken based at least in part on the one or more parameters. For example, embodiments are contemplated in which a particular catheter device is selected based on the one or more parameters, such as distance from the surface of the skin to the vein and suggested catheter length, and the selected catheter device is automatically ordered.

In some embodiments, a plurality of instructions may be generated and provided to a medical personnel through the user device 128 or the device 120 to guide the medical personnel through the ultrasound and insertion procedure. In a particular example, a medical personnel may first be instructed to position a probe of the ultrasound device 110 on a forearm of a patient such that a potential insertion site may be identified within the patient's forearm. Subsequent instructions may be provided to reposition the ultrasound device 110, for example, to locate another potential insertion site or to provide a better image of the first potential insertion site. Further, in some embodiments, the medical personnel may be instructed to move the ultrasound device 110 to another portion of the patient's body responsive to determining that one or more potential insertion sites in the patient's forearm are below a predetermined insertion score threshold. For example, the medical personnel may be instructed to place the ultrasound device 110 on the patient's other arm.

FIG. 2 illustrates an example of an ultrasound 200 relating to some embodiments. In some embodiments, the ultrasound 200 includes one or more ultrasound images. For example, the ultrasound 200 may include a biplane ultrasound view comprising both a cross-sectional ultrasound image 202 and a side-view ultrasound image 204, as shown. Alternatively, or additionally, in some embodiments, a singular image may be displayed. In some embodiments, the cross-sectional ultrasound image 202 shows a cross-sectional visualization of a vein 206 of a patient and a visualization of a catheter 208. In some embodiments, each of the cross-sectional ultrasound image 202 and the side-view ultrasound image 204 may be captured by and received from the ultrasound device 110. In some embodiments, the catheter 208 may be the catheter 102, as shown in FIG. 1. Further, in some embodiments, only a portion of the catheter 208 may be visible in the ultrasound 200. For example, only the insertion portion 104 may be visible within the cross-sectional ultrasound image 202.

In some embodiments, the ultrasound 200 may be generated for display within a display of the user device 128 or the device 120. For example, the ultrasound 200 may be viewed by a medical personnel in real-time prior to and during a catheter insertion procedure. Further, in some embodiments, the ultrasound images may be augmented with additional information. For example, one or more anatomical structures may be identified within the ultrasound image and indicated via augmentation of the display.

In some embodiments, a catheter to vein ratio may be deduced or estimated from the cross-sectional ultrasound image 202. For example, the catheter to vein ratio may include a comparison of the catheter diameter to the inner vein diameter of the vein. In some embodiments, a specific type of catheter 208 may be selected to achieve a suitable catheter to vein ratio.

In some embodiments, the side-view ultrasound image 204 shows side view visualizations of the vein 206 and the catheter 208. In some embodiments, the side-view ultrasound image 204 may visually indicate an angle of insertion of the catheter 208. In some embodiments, the cross-sectional ultrasound image 202 and the side-view ultrasound image 204 may be received and displayed in real-time to aid a user in inserting and positioning the catheter 208. Additionally, in some embodiments, markings may be included showing a measurement scale adjacent to the cross-sectional ultrasound image 202 and the side-view ultrasound image 204 4. For example, incremental length measurements may be included on a side of the ultrasound 200, as shown.

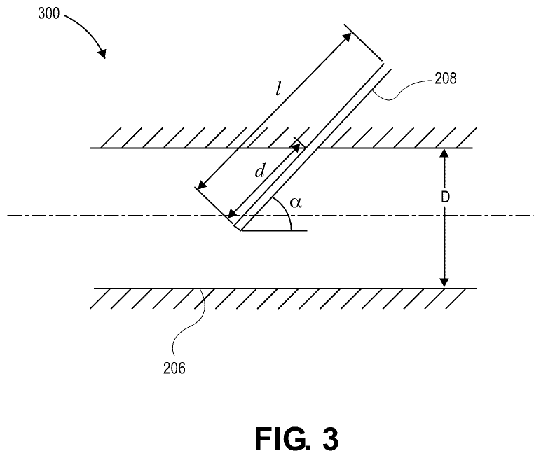

FIG. 3 illustrates an exemplary operational environment of an insertion region 300 relating to some embodiments. In some embodiments, the insertion region 300 includes a vein 206 or other type of blood vessel. The catheter 208 may be inserted into the vein 206, as shown. In some embodiments, the catheter 208 has a length l and is inserted at an angle α. In some such embodiments, the angle α may be measured from an axis normal to the cross section of the vein 206, as shown. Additionally, the catheter 208 may be inserted at a depth d into the vein 206. The vein 206 may have a diameter D. Accordingly, in some embodiments, an optimal depth d of the catheter 208 may depend on the diameter D of the vein 206.

In some embodiments, any of the insertion parameters described above may be suggested based on parameters of the vein 206. For example, the length of the catheter, the angle of the catheter, and the depth of the catheter into the vein may be suggested based on the size of the vein and a blood flow velocity rate of the vein. In some embodiments, the parameters may be determined to achieve a suggested hemodilution ratio for proper administration of a medication into the patient's blood stream.

In some embodiments, a length for the catheter may be selected based on a depth of vein and angle of insertion for the catheter using trigonometric functions. For example, the Pythagorean theorem may be employed to calculate a suitable length of the catheter based on the depth of center for the vein and the travel across the length of the vein based on the angle of insertion.

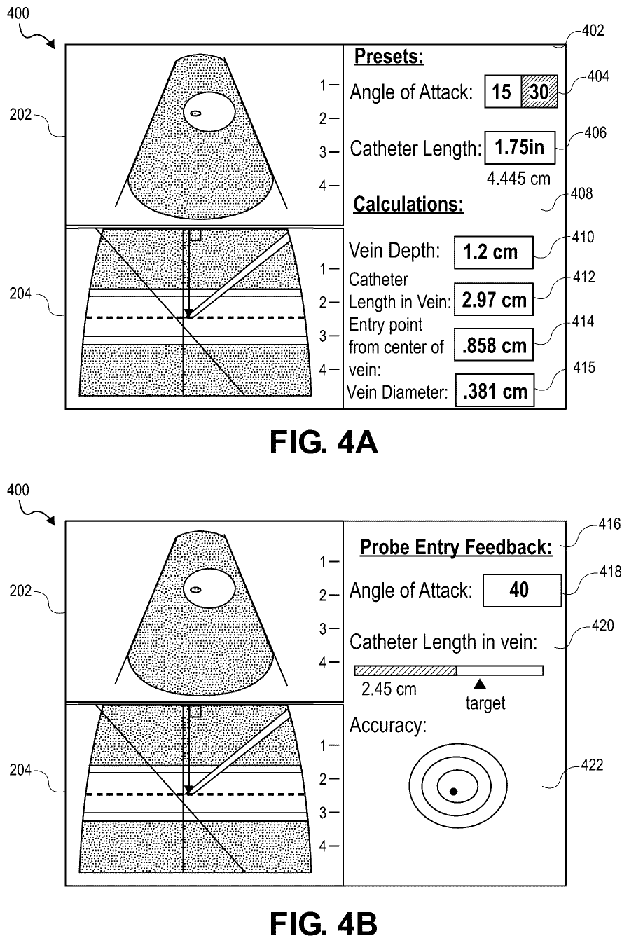

FIG. 4A illustrates an example of a user interface 400 relating to some embodiments. In some embodiments, the user interface 400 may be displayed on one or more user devices during insertion and positioning of the catheter 208. For example, the user interface 400 may be displayed as part of an application running on a mobile device or another suitable computing device. In some such embodiments, the user interface 400 may include the cross-sectional ultrasound image 202 and side-view ultrasound image 204 of the ultrasound 200. Accordingly, the user interface 400 may be used to aid in inserting and positioning the catheter 208. Additionally, in some embodiments, the user interface 400 may be displayed prior to insertion of the catheter 208 such that the user interface 400 may be used to select a catheter appropriate and/or optimized for a specific vein. For example, the ultrasound 200 may be used to select an optimally sized catheter.

In some embodiments, the user interface 400 includes indicators of one or more preset parameters 402. For example, an angle of insertion 404 and a catheter length 406 may be listed on the user interface 400, as shown. The angle of insertion 404 may refer to the actual angle or the recommended target angle of the catheter 208 within the vein 206. In some embodiments, the angle of insertion 404 may be the same as the angle α shown in FIG. 3. In some embodiments, the angle of insertion 404 and the catheter length 406 may be manually selected by a user. Further, in some embodiments, the angle of insertion 404 and/or the catheter length 406 may be automatically determined, for example, based on a catheter 208 selected by the user or system and on vein 206.

In some embodiments, the user interface 400 further comprises indications of one or more calculated parameters 408. For example, the one or more calculated parameters 408 may include any of a vein depth 410, catheter length in vein 412, entry point from center of vein 414, and a vein diameter 415 may be listed on the user interface 400, as shown. In some embodiments, the one or more calculated parameters 408 may be calculated automatically, for example, based at least in part on data from the ultrasound 200. In some embodiments, a suggested catheter length in vein is approximately two thirds of the length of the catheter.

In some embodiments, additional mark-up may be included in the user interface 400 overlayed onto the cross-sectional ultrasound image 202 and the side-view ultrasound image 204, as shown. In some embodiments, the additional mark-up may highlight the angle of the catheter 208 and the center of the vein 206, as shown.

FIG. 4B illustrates another example of user interface 400 relating to some embodiments. In some embodiments, the user interface 400 includes one or more indicators of probe entry feedback 416. For example, the probe entry feedback 416 may include a measured angle of insertion 418, a measured catheter length in vein 420, and an accuracy 422. In some such embodiments, the measured angle of insertion 418, the measured catheter length in vein 420, and the accuracy 422 may be determined based on any combination of the ultrasound data and/or sensors associated with the catheter 208. In some embodiments, the angle of insertion 418 may be color coded to thereby indicate an acceptability of the angle of insertion. For example, if the angle of insertion is outside of an acceptable range, the angle of insertion 418 may be shown in red and if the angle of insertion 418 is within an acceptable range, the angle of insertion 418 may be shown in green. In some embodiments, any of the probe entry feedback parameters may be color coded to indicate an acceptability of said parameters to aid in positioning of the catheter 208. In some embodiments, the accuracy 422 may be determined relative to a center of the vein 206. In some embodiments, the measured catheter length in vein 420 may be shown relative to a target length, as shown, to aid an operator in inserting and positioning the catheter 208. Accordingly, in some embodiments, imagery may be included showing the placement of the catheter 208 within the vein 206, as shown.

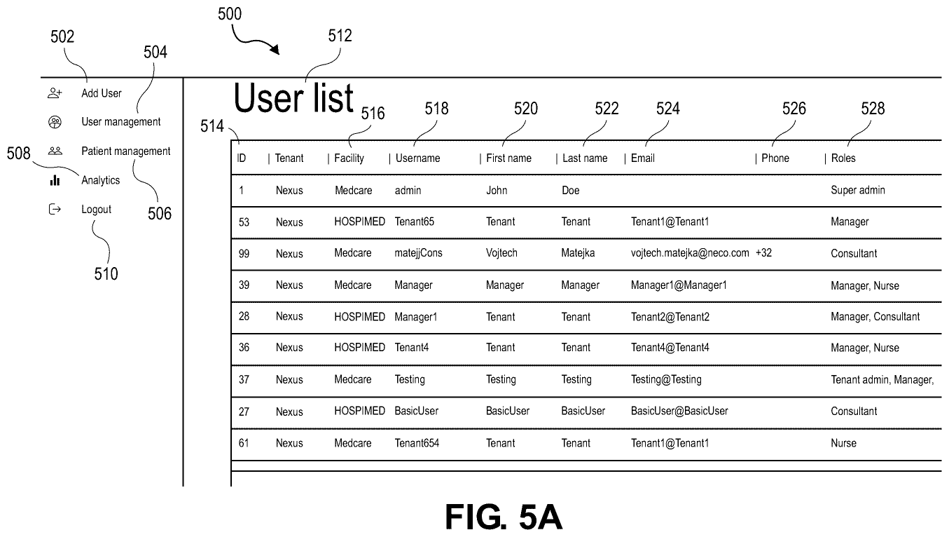

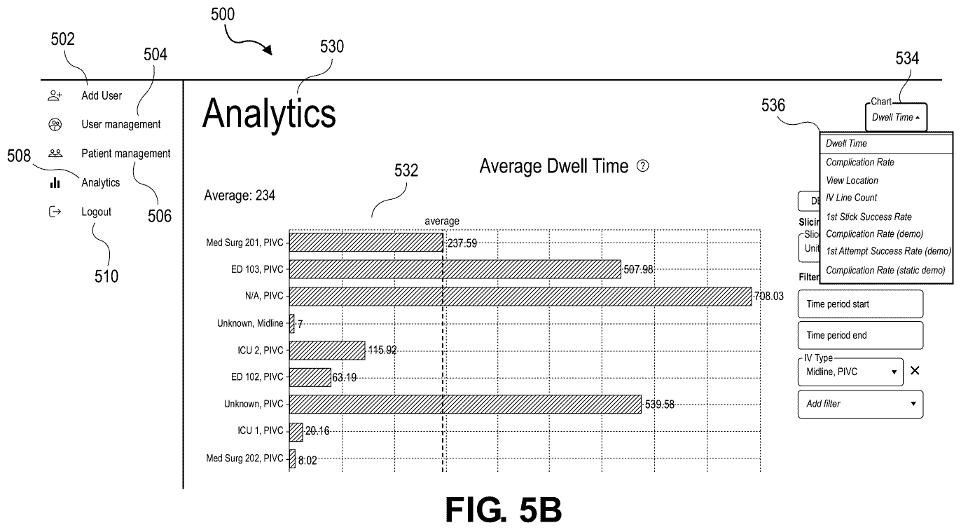

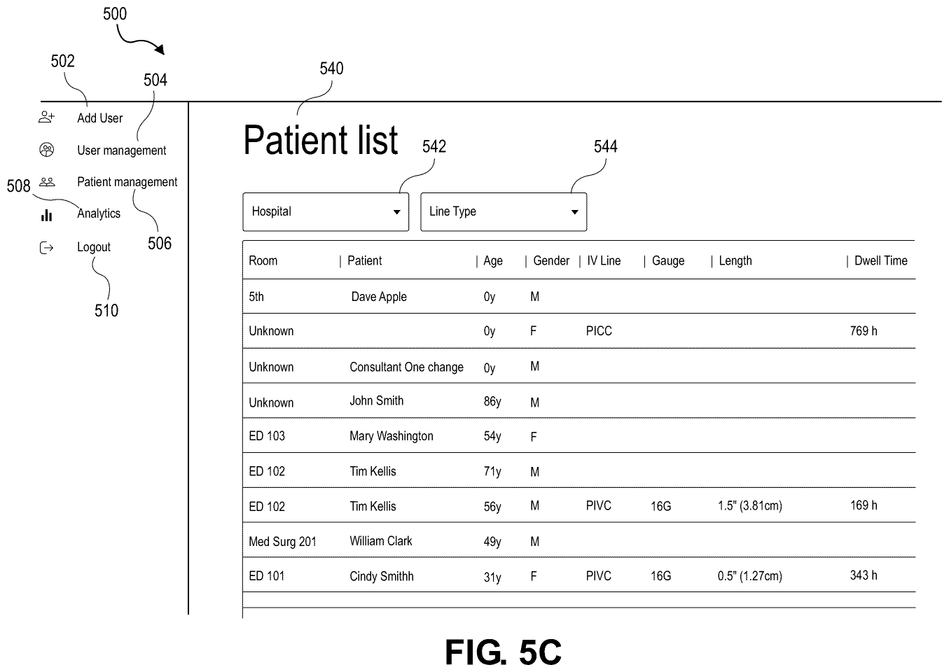

FIG. 5A illustrates an example of a user interface 500 relating to some embodiments. In some embodiments, the user interface 500 may be displayed on a user device as part of an application running on said user device. In some such embodiments, the user interface 500 may be associated with the user interface 400, as shown in FIGS. 4A and 4B. Alternatively, or additionally, in some embodiments, separate user interfaces may be included for various functionalities. For example, a first application may be associated with aiding with catheter insertion and positioning in real-time while another application may be associated with monitoring and comparing catheter data over time.

In some embodiments, the user interface 500 may include a variety of selectable pages and actions. For example, the user interface 500 may include an add user selection option 502, a user management selection option 504, a patient management selection option 506, an analytics selection option 508, and a logout selection option 510. In some such embodiments, each of the selection options 502-510 may be associated with a different user interface page or action. For example, the add user selection option 502 may be used to access an add user interface page operable to add a new user to a user database of the application. Further, the user management selection option 504 may be used to edit information within the user database such as user parameters and/or to remove user information from the user database.

In some embodiments, the patient management selection option 506 may be used to access a patient management interface page operable to access and edit patient data stored in a patient data store. Embodiments are contemplated in which patient data may be automatically stored within the patient data store. For example, ultrasound data, as described above may be automatically recorded and stored within the patient data store and associated with a patient profile of the patient. Further, in some embodiments, the analytics selection option 508 may be used to access an analytics interface page operable to view and monitor analytics data associated with a plurality of patients.

In some embodiments, the user interface 500 includes a user list 512, as shown. For example, the user list 512 may be accessed as part of the user management page accessed via the user management selection option 504. The user list 512 may include various user information. For example, the user list 512 may include a set of user information relating to a respective plurality of users, such as, a user ID 514, a tenant name, a facility name 516, a username 518, a first name 520, a last name 522, an email address 524, a phone number 526, and one or more user roles 528. In some embodiments, the user information may be collected when a new user is added. Further, in some embodiments, the user interface 500 allows the user information to be added and updated after a user account is established.

FIG. 5B illustrates another example of user interface 500 relating to some embodiments. In some embodiments, an analytics page 530 may be accessed within the user interface 500, as shown. For example, the analytics page 530 may be accessed via the analytics selection option 508. In some embodiments, the analytics page 530 includes analytics data associated with the plurality of patients. For example, the analytics page 530 may include one or more charts such as data analytics charts 532 populated with patient data relating to the plurality of patients. In some embodiments, the data analytics chart 532 includes average dwell time of the plurality of patients, as shown. In some embodiments, a plurality of different types of charts may be selected from using a chart selection actuator 534. For example, in some embodiments, the chart selection actuator 534 may generate a list of chart types 536 upon selection. In some embodiments, the chart types include any combination of dwell time, complication rate, vein location, IV line count, vein diameter, vein depth, IV/catheter size, IV/catheter gauge, IV/catheter length, and first stick success rate. However, it should be understood that, in some embodiments, additional chart types are also included.

FIG. 5C illustrates yet another example of user interface 500 relating to some embodiments. In some embodiments, a patient list 540 may be accessed within the user interface 500, as shown. In some such embodiments, the patient list 540 may be accessed via the patient management selection option 506. In some embodiments, one or more filters may be applied to the patient list 540. For example, a hospital filter 542 and a line type filter 544 may be applied for a more narrowly targeted a patient search. In some embodiments, the patient list 540 is populated with patient information for the respective plurality of patients. For example, the patient list 540 may include any of a room number, a patient name, a patient age, a patient gender, an IV line type, an IV gauge, an IV length, a dwell time, and an insertion time, as shown. In some embodiments, the patient list 540 may be used to track and manage a plurality of Ws for a respective plurality of patients. In some embodiments, information in the patient list 540 may be stored and retrieved at a later time to select a subsequent catheter. For example, if a specific type of catheter in a specific vein of the patient has had success historically based on the patient list 540, then a similar catheter type may be selected for future insertions. Conversely, if a catheter does not have success, that catheter type may be avoided for future insertions.

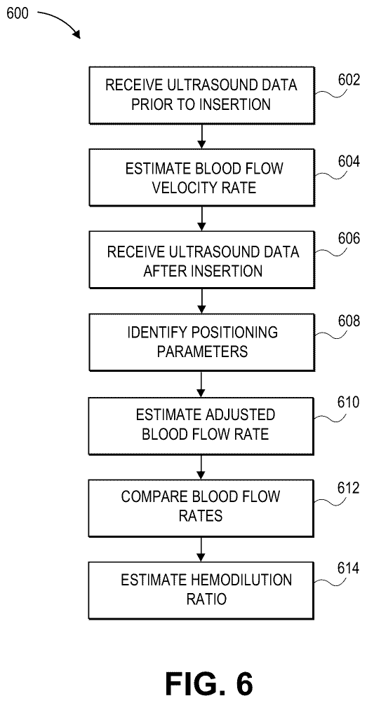

FIG. 6 illustrates an example of a method 600 of inserting an intravenous catheter into a blood vessel of a patient relating to some embodiments. In some embodiments, at least a portion of the steps of the method 600 may be performed by at least one processor, such as, for example, the processor 122. Further, in some embodiments, one or more steps may be performed by a user or operator of the catheter 208. Additionally, embodiments are contemplated in which any number of the steps of method 600 may be performed automatically.

At step 602, a first set of ultrasound data in received prior to insertion of a catheter 208. In some embodiments, the first set of ultrasound data may include ultrasound imagery relating to one or more veins of the patient. Accordingly, the ultrasound data may be used to select a vein for insertion of the catheter. In some embodiments, one or more edges of the vein may be detected based on the received ultrasound data. At step 604, a blood flow velocity rate of the vein may be estimated based at least in part on the first set of ultrasound data. The blood flow velocity rate may be an initial volumetric blood flow velocity rate calculated prior to insertion of the catheter. In some embodiments, a volumetric blood flow velocity rate may be calculated by multiplying the velocity of the blood flow by the value π and the vein diameter.

In some embodiments, any combination of the first set of ultrasound data and the estimated blood flow velocity rate may be used to select an appropriately sized catheter for the vein. For example, in some embodiments, a size of the vein may be identified from the first set of ultrasound data. The diameter of the vein may be used to select an appropriately sized catheter. For example, in some embodiments, the catheter may be selected to achieve a desired catheter to vein ratio, as described above. For example, in some embodiments, a catheter diameter may be selected to compliment a diameter of the vein. In some embodiments, a suggested catheter length may be determined and calculated based on the ultrasound data of the vein. In some embodiments, the suggested catheter parameters, such as catheter length and catheter gauge size, may be retrieved from a catheter/vein chart based on one or more vein parameters.

In some embodiments, one or more suggested positioning parameters or other catheter parameters associated with the intravenous catheter may be suggested based at least in part on the first set of ultrasound data. In some embodiments, the one or more suggested positioning parameters may be displayed to a user within a user interface such as the user interface 400 or user interface 500 described above.

At step 606, a second set of ultrasound data is received after insertion of the catheter 208 into the vein 206. At step 608, one or more positioning parameters of the catheter 208 are identified. In some embodiments, the one or more positioning parameters may be identified based at least in part on the second set of ultrasound data including any of an angle of insertion, depth in vein, accuracy to center of vein, and a number of other positioning parameters relating to the insertion of the catheter 208 within the vein 206.

At step 610, an adjusted blood flow velocity rate is estimated based at least in part on the second set of ultrasound data after insertion of the catheter 208 within the vein 206. In some embodiments, the adjusted blood flow velocity rate comprises an infusion flow rate associated with the blood flow velocity rate after insertion of the catheter. Accordingly, the infusion flow rate may be calculated similarly to the volumetric blood flow velocity rate described above. At step 612, the adjusted blood flow velocity rate is compared to the initial blood flow velocity rate. In some embodiments, the blood flow velocity rates are compared to monitor the effects of the inserted catheter on the blood flow velocity rate. For example, in some cases, the insertion of the catheter impedes the blood flow velocity rate. However, it may be desirable that the effects of the catheter insertion on the blood flow velocity rate are minimized. Accordingly, comparing the blood flow velocity rates allows for adjustment of the catheter positioning to thereby optimize the positioning of the catheter such that the blood flow velocity rate is not significantly impeded.

At step 614, a hemodilution ratio is estimated. In some embodiments, the hemodilution ratio may be estimated based at least in part on the ultrasound data and the estimated blood flow velocity rate. In some embodiments, the hemodilution ratio may be calculated by dividing the initial volumetric blood flow velocity rate by the infusion blood flow velocity rate. Accordingly, in some embodiments, the hemodilution ratio may comprise a metric of comparison of the blood flow velocity rate after insertion of the catheter. In some embodiments, the hemodilution ratio represents the dilution of medication into the blood stream. In some embodiments, a 3:1 ratio is desirable.

In some embodiments, various parameters may be monitored associated with insertion of the catheter. For example, a catheter to vein ratio may be determined based on comparing the catheter gauge and selected vein diameter. For example, in some embodiments, a catheter to vein ratio of 40% or less is desirable. Further, a suggested catheter length may be determined based on vein depth and angle of insertion of the catheter. In some embodiments, it may be desirable that 65% of the catheter is inserted into the vein.

In some embodiments, the ultrasound data may be stored and used in a simulation training program. For example, a catheter insertion simulation may be generated using historical ultrasound data. Accordingly, the catheter insertion simulation may be used to train medical personnel in catheter insertion.

### Machine Learning

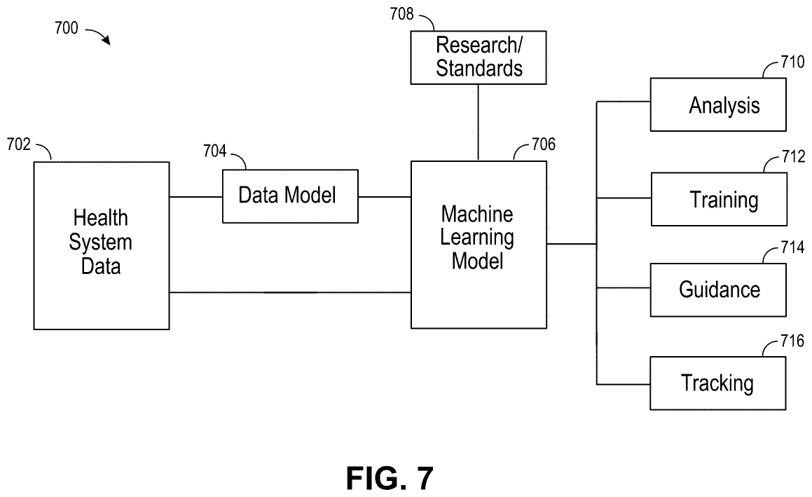

FIG. 7 illustrates an exemplary system diagram of a data system 700 relating to some embodiments of the present disclosure. In some embodiments, the components described with respect to the data system 700 may comprise any combination or one of hardware and software components. For example, any of said components may be included as computer or server hardware. Further, in some embodiments, the components may be included as software configured to be executed on at least one processor. For example, any of the components may comprise non-transitory computer-readable media configured to be executed on at least one processor such as an executable application.

The data system 700 may comprise health system data store 702, as shown. The health system data store 702 may include health system data from any of a plurality of health system sources such as historical ultrasound data from ultrasound providers, medical patient data, policies and procedures from health systems, user data, and other suitable health system data.

The health system data store 702 may be communicatively coupled to a data model 704, as shown. For example, the data model 704 may be generated based at least in part on the health system data from the health system data store 702. In some embodiments, the data model 704 includes training data for training one or more machine learning models. In some embodiments, the data model 704 comprises any of dwell time analysis data, medical complication data, care and maintenance data, as well as medical personnel productivity data.

At least one machine learning model such as machine learning model 706 may be included. For example, the machine learning model 706 may include a computer vision machine learning model configured to identify one or more anatomical structures within a set of real-time ultrasound data. For example, the machine learning model 706 may be trained with training data from the data model 704 or from data received directly from the health system data store 702. Alternatively, or additionally, in some embodiments, the machine learning model 706 may comprise any combination of an analysis and recommendation model, a training model, a guidance model, and a tracking model.

In some embodiments, the machine learning model 706 may be communicatively coupled to one or more research and standards data stores 708. The research and standards data stores may store a plurality of information relating to medical research and medical standards such as clinical research data, medical evidence, and medical information from other data sources.

The machine learning model 706 may output any combination of analysis 710 configured to analyze and provide recommendations in real-time to medical personnel, training 712 configured to train medical personnel, guidance 714 configured to provide real-time instructions and guidance to medical personnel, and tracking 716 configured to monitor medical procedures and provide real-time care and maintenance tracking.

In some embodiments, the machine learning model is configured to determine one or more parameters for the insertion procedure. For example, one or more suggested parameters for the catheter may be selected based on the real-time ultrasound data. Further, the machine learning model may be configured to determine a suitable angle of insertion and position for insertion of the catheter based on the ultrasound data. Further still, in some embodiments, the machine learning model is configured to select a suitable vein for insertion based on the real-time ultrasound data.

In some embodiments, the machine learning model comprises a generative artificial intelligence model configured to generate training content based on collected ultrasound data. For example, the machine learning model may analyze historical training of medical personnel and medical personnel performance in order to provide medical training content optimized for successful performance. As a specific example, if a particular image or instruction that was presented to a plurality of medical personnel during training and those medical personnel received a high performance score, that image or instruction may be selected for inclusion in subsequent training content. Additionally, the machine learning model utilizing generative artificial intelligence may be operable to access the one or more research and standards data stores 708 to check and improve training content based on information on the one or more research and standards data stores 708. In some embodiments, a persistent set of medical personnel training content may be stored and continuously updated based on acquired medical evidence and ultrasound data over time. Accordingly, training content may improve over time and methodology may be updated and improved over time to enhance and standardize catheter insertion procedures.

### Computer Vision

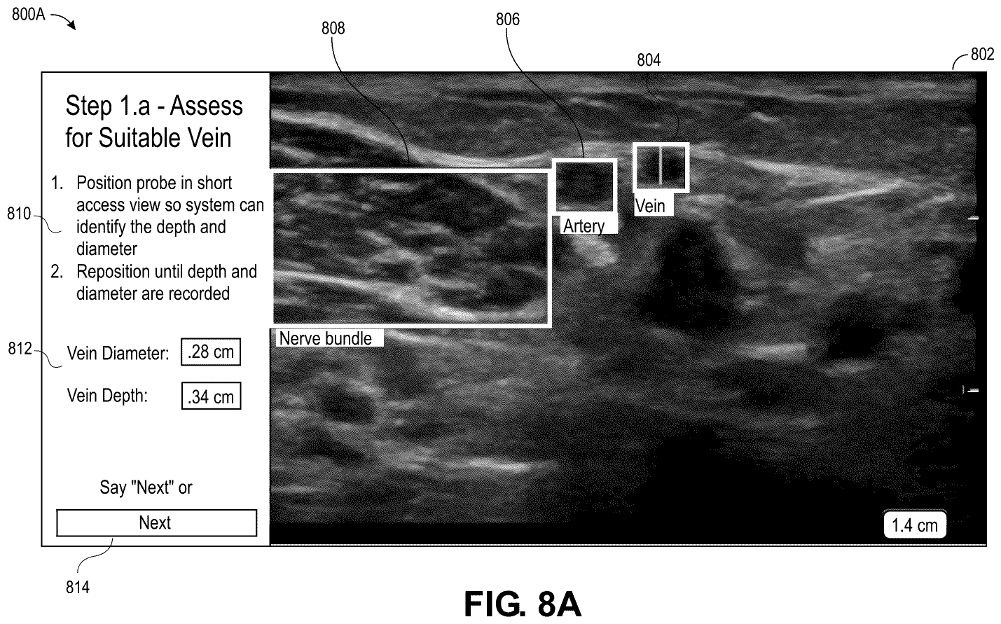

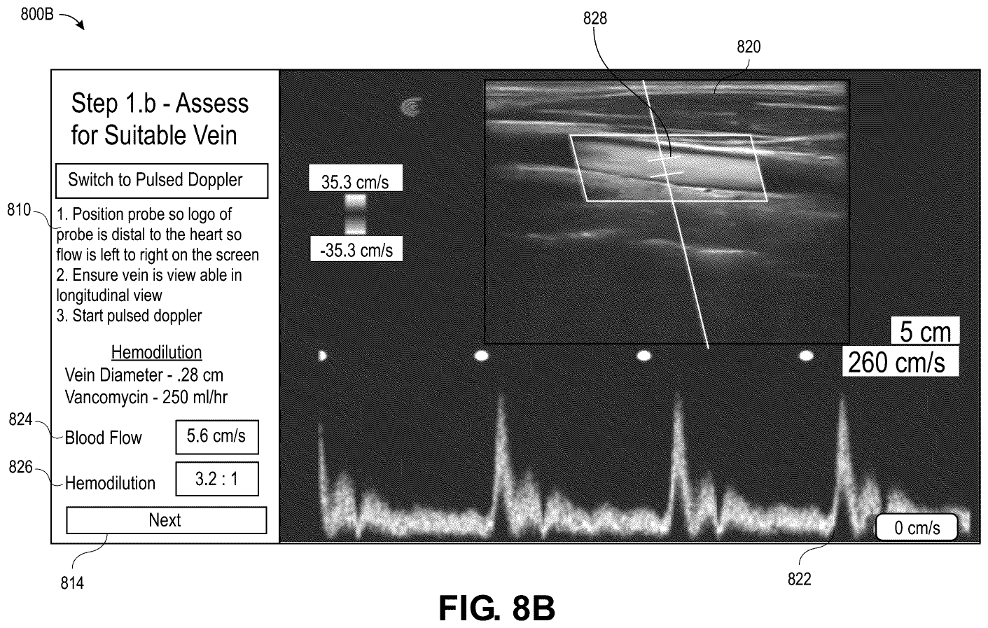

FIG. 8A illustrates an example of a user interface 800A relating to some embodiments of the present disclosure. In some embodiments, the user interface 800A may be configured for display within a display of one or more user devices such as, a computer screen, a mobile phone screen, or a tablet display screen. Further, embodiments are contemplated in which at least a portion of the user interface 800A may be included within an augmented reality overlay or virtual reality display. For example, the user interface 800A may be displayed within an augmented reality display device configured to be worn by a medical personnel such as a nurse performing an insertion procedure of an intravenous catheter.

In some embodiments, a suggested target slope may be generated for display and overlaid onto the real-time ultrasound image. For example, the suggested target slope may be selected based at least in part on one or more parameters associated with the insertion site and may achieve a low angle of insertion to improve blood flow after insertion. Accordingly, the medical personnel is able to visually compare the target slope angle and the actual angle of insertion of the catheter needle during insertion.

The user interface 800A includes a real-time ultrasound image 802, as shown. For example, the real-time ultrasound image 802 may include an ultrasound image captured by the ultrasound device 110 of a potential catheter insertion site of a patient. As such, the real-time ultrasound image 802 may be captured and displayed prior to selection and/or insertion of the catheter 102.

In some embodiments, one or more anatomical structures are identified within the real-time ultrasound image 802. For example, a computer vision algorithm may be used to identify anatomical structures within the real-time ultrasound image 802 and provide an indication to medical personnel to locate and indicate said anatomical structures. The anatomical structures may include any combination of one or more veins 804, one or more arteries 806 (or other blood vessels), and one or more nerve bundles 808. Further, in some embodiments, other anatomical structures may be identified such as any of valves and muscle tissue. The identification of the anatomical structures may aid in guiding the medical personnel through successful vein selection for insertion.

In some embodiments, the anatomical structures may be highlighted or otherwise visually indicated through augmentation of the ultrasound image. For example, in some embodiments, each type of anatomical structure may be highlighted with a different color within the user interface 800A such that the medical personnel is able to distinguish between structures and other objects present in the ultrasound image. Further, in some embodiments, the information indicative of the identified anatomical structures from the real-time ultrasound image 802 may be transmitted to one or more other machine learning models for automatic selection and/or suggestion of a particular vein for insertion. The computer vision algorithm may be further configured to determine one or more parameters associated with the identified anatomical structures and the intravenous catheter such as, for example, diameter, depth, blood flow velocity, angle of insertion, vein purchase, and catheter tip location.

In some embodiments, additional information may be included within the user interface 800A. For example, in some embodiments, the user interface 800A provides real-time instructions 810 configured to guide a medical personnel through an intravenous catheter insertion procedure. As an example, the real-time instructions 810 may include a first instruction for guiding the medical personnel through properly positioning a probe of the ultrasound device 110 and a second instruction for subsequent repositioning of the probe. The real-time instructions 810 may be provided and updated by the user interface 800A in real-time while the medical personnel prepares for and performs the insertion procedure.

Additionally, in some embodiments, the user interface 800A further includes one or more real-time parameters 812. For example, the real-time parameters may include parameters measured, calculated, or estimated from the ultrasound data such as a vein diameter, vein pressure, and vein depth of a selected vein. Further, in some embodiments, the user interface 800A comprises one or more interface actuators such as interface actuator 814 for allowing the medical personnel or other users to interact with the user interface 800A. For example, the interface actuator 814 may comprise a next interface button configured to proceed to a subsequent user interface or set of instructions. Here, the user may select the next button once the listed instructions have been completed to proceed with further guidance from the user interface 800A.

FIG. 8B illustrates an example of a user interface 800B relating to some embodiments of the present disclosure. In some embodiments, similar to user interface 800A, the user interface 800B may be configured for display within a display of one or more user devices such as, a computer screen, a mobile phone screen, or a tablet display screen. Further, embodiments are contemplated in which at least a portion of the user interface 800B may be included within an augmented reality overlay or virtual reality display. For example, the user interface 800B may be displayed within an augmented reality display device configured to be worn by a medical personnel such as a nurse performing an insertion procedure of an intravenous catheter. In some embodiments, user interface 800B may be associated with user interface 800A, as described above. For example, user interface 800A and user interface 800B may be the same user interface but may be displayed at different times.

In some embodiments, the user interface 800B includes a lengthwise view 820 of a selected vein. Accordingly, the medical personnel may be able to visualize an angle and positioning of the vein within the body of the patient. As such, the lengthwise view 820 may be utilized to assess the suitability of the vein for intravenous catheter insertion.

Similar to user interface 800A, the user interface 800B may include real-time instructions 810. For example, instructions for positioning the ultrasound probe, ensuring a suitable view of the vein, and initializing a pulsed doppler signal transmission. Accordingly, in some embodiments, a pulsed doppler signal data 822 may be included within the user interface 800B such that the medical personnel is able to simultaneously view the lengthwise view 820 of the selected vein and the pulsed doppler signal data 822.

Additionally, the user interface 800B may include one or more real-time parameters such as the vein diameter and vein depth. In some embodiments, the user interface 800B includes a blood flow rate 824 and a hemodilution rate 826. The blood flow rate 824 and hemodilution rate 826 may be determined based at least in part on the real-time ultrasound data using any suitable method as described above.

In some embodiments, a suggested angle of insertion 828 for insertion of the catheter is included, as shown. In some embodiments, the angle of insertion may be determined based at least in part on an angle of the vein. For example, a default angle of insertion may be about 15 degrees from the lengthwise axis of the vein. However, in some embodiments, the angle of insertion may be adjusted based on a variety of factors.

### Training and Monitoring

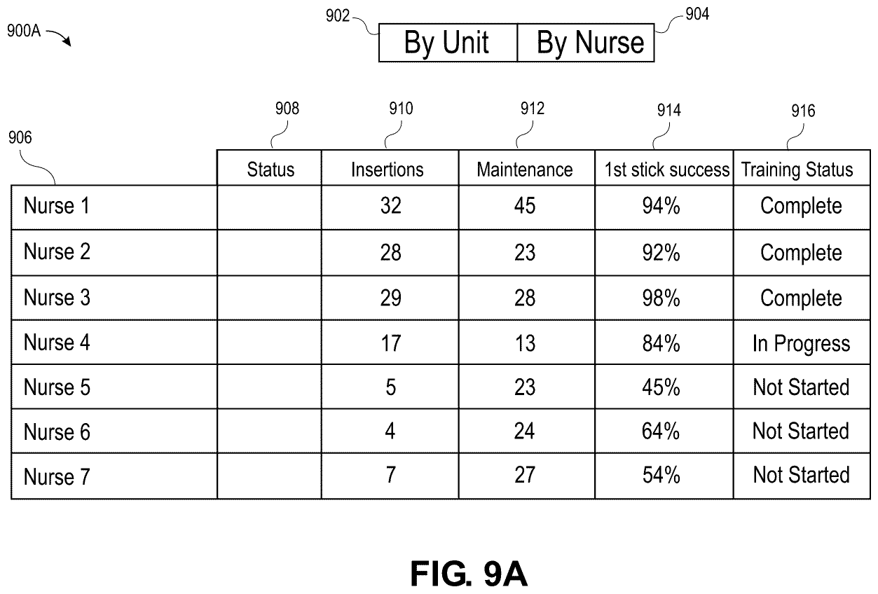

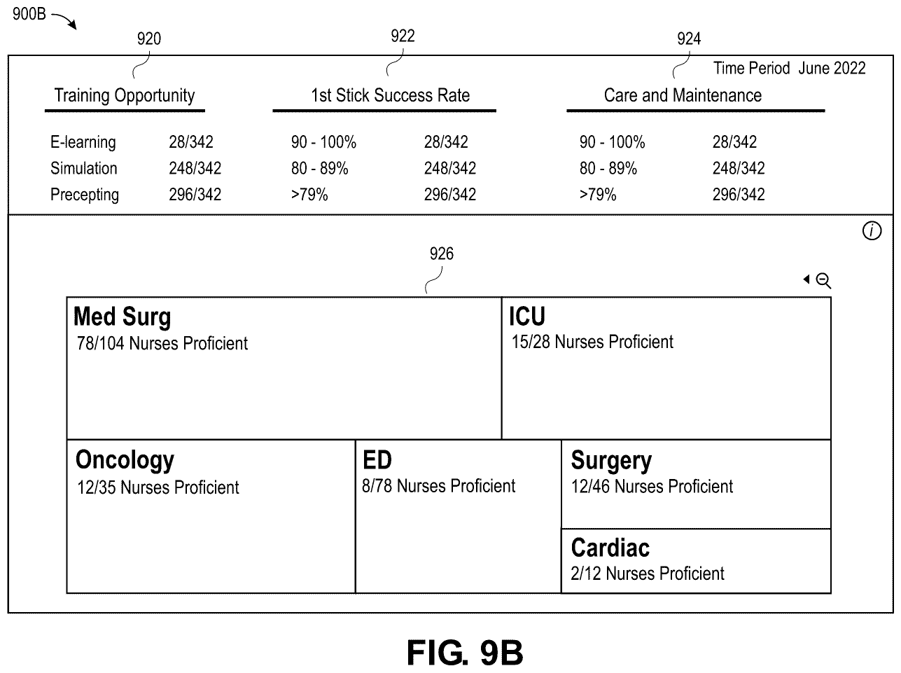

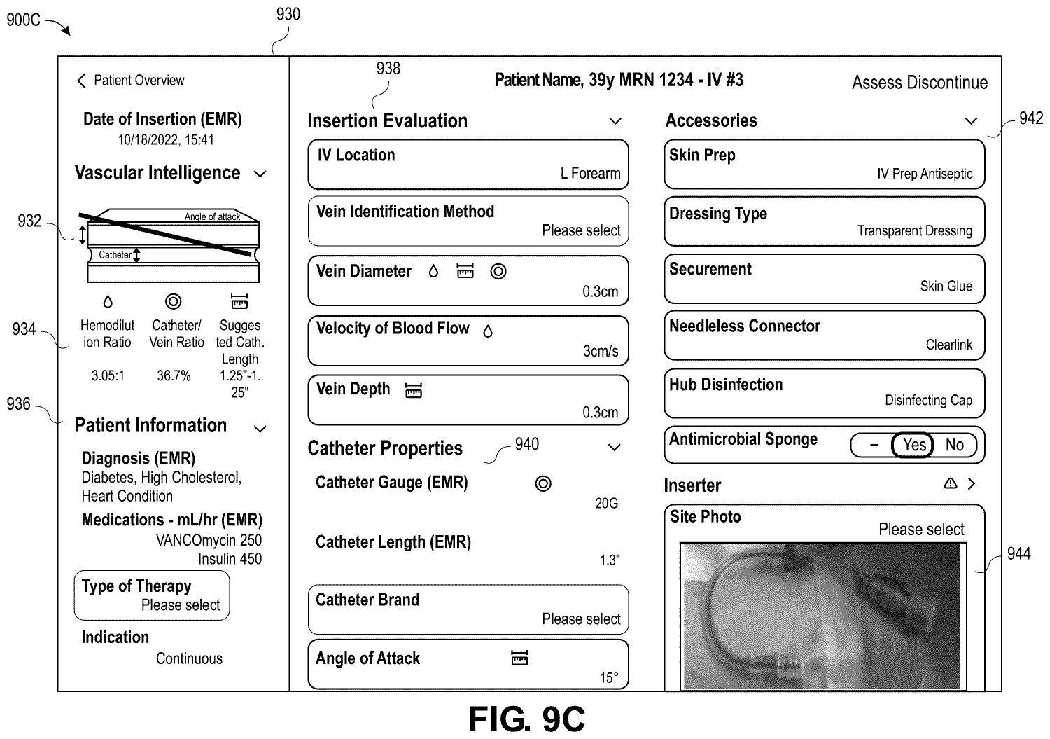

FIG. 9A illustrates an example of a table 900A showing catheter insertion records corresponding to a plurality of medical personnel. In some embodiments, the table 900A may be included within a user interface such as a training and monitoring interface. Said user interface may include selection options for displaying recorded information based on either of a medical staff unit 902 or by individual medical personnel 904.

The table 900A includes a first column listing a plurality of medical personnel 906 for which information has been recorded. The table 900A further includes a current status 908, a number of insertions 910, a number of maintenance operations 912, a percentage of first insertion success 914, and a training status 916 for each respective medical personnel of the plurality of medical personnel 906. In some embodiments, the current status 908 represents an overall proficiency of the medical personnel in catheter insertion operations.

FIG. 9B illustrates an example of a user interface 900B relating to some embodiments of the present disclosure. The user interface 900B may include information relating to a plurality of medical personnel and medical staff units, as shown, such as training completion information 920 showing a number of medical personnel who have completed a particular training program, first stick success rate information 922 showing a number of medical personnel who have recorded a particular percentage of first insertion success in catheter insertion operations, and care and maintenance information 924 showing a number of medical personnel who have completed a particular percentage of care and maintenance operations.

The user interface 900B may be presented to a user such as a medical administrator to monitor and review information relating to the plurality of medical personnel. Accordingly, the medical administrator may use the user interface 900B to consider a proficiency of the overall medical staff or particular individuals relating to intravenous catheter insertion procedures. Further, the user interface 900B may be used to determine which medical personnel are equipped to perform said insertion procedures and which staff units should be associated with insertion procedures. Additionally, in some embodiments, the information depicted in table 900A and user interface 900B may be provided to the machine learning model to automatically determine updates to training and to properly assign insertion procedures among available medical personnel.

In some embodiments, the user interface 900B further comprises a staff unit breakdown diagram 926 showing a breakdown of each staff unit of medical personnel and indicating a number of medical personnel that have achieved a proficient score for catheter insertion out of a total number of medical personnel.

In some embodiments, further machine learning and artificial intelligence techniques may be employed to improve training for medical personnel. For example, stored ultrasound data from a previous insertion procedure may be reviewed by a machine learning model to identify errors and provide improvement recommendations to medical personnel.

In some embodiments, a machine learning model or other computing or control device is operable to generate a suggestion to retrain one or more medical personnel based on the data presented in either of table 900A or user interface 900B. For example, if a first insertion success 914 for a particular medical personnel is below a predetermined threshold, that medical personnel may be selected for retraining. Further, in some embodiments, the machine learning model may be trained to identify a particular error or action that lead to the low success rate of the medical personnel and cater training content around that particular error or action. Further, in some embodiments, one or more medical personnel may be selected to perform a particular insertion procedure based on the medical personnel training and performance data. For example, if a patient record indicates that a patient has had difficulties in insertion in the past one or more medical personnel with insertion performance above a predetermined performance threshold may be automatically selected to perform the insertion procedure. In some embodiments, medical personnel selection may be selected based further on medical personnel availability.

FIG. 9C illustrates an example of a user interface 900C including an insertion record 930 relating to some embodiments of the present disclosure. The user interface 900C shows a plurality of insertion parameters recorded for an insertion procedure. In some embodiments, the insertion parameters may be added and stored automatically. Alternatively, or additionally, the parameters may be input manually for example, by a medical personnel typing into the user interface or via a hands off verbal input, as described above.

In some embodiments, the insertion record 930 includes a visual insertion representation 932 showing a catheter needle and insertion region of the patient, as well as indications of the vein depth, vein diameter, and angle of insertion of the catheter. Further, the insertion record 930 may include insertion parameters 934 indicating a hemodilution ratio, catheter vein ratio, and suggested catheter length. In some embodiments, patient information 936 is included on the insertion record 930, as shown.

In some embodiments, the insertion record 930 includes an insertion evaluation portion 938 including insertion parameters such as IV location, vein identification method, vein diameter, velocity of blood flow, vein depth, etc., as well as a catheter properties portion 940 including catheter information associated with the selected catheter such as a catheter gauge, catheter length, catheter brand, and an angle of insertion of the catheter.

Additionally, in some embodiments, the insertion record 930 further comprises an accessories portion 942 including additional information associated with the insertion procedure such as a skin prep technique, dressing type, securement, needless connector, and Hub disinfection technique, and indication of whether an antimicrobial sponge was used. Further, an insertion site photo 944 may be included, as shown, to provide an actual visual representation of the insertion region after the insertion procedure.

The insertion record 930 described above and information included therein may be stored within a medical data store. For example, in some embodiments, the insertion record 930 may be added to a patient profile within a medical system database such that the insertion record 930 may be reviewed at a later time in regard to the patient, for example, prior to a subsequent insertion procedure. Similarly, in some embodiments, the insertion record 930 may be added to a medical personnel profile to monitor performance of the medical personnel.

### Methods of Operation

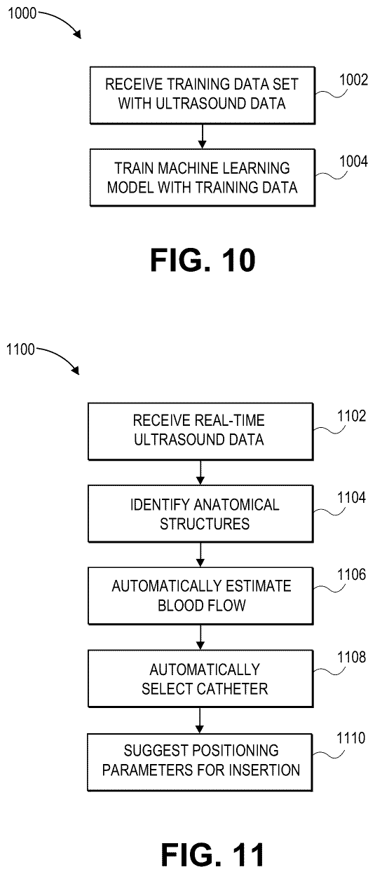

FIG. 10 illustrates an example of a method 1000 providing a machine learning model relating to some embodiments of the present disclosure. In some embodiments, the method 1000 may be performed by at least one processor executing a set of non-transitory computer-readable instructions. For example, one or more steps of the method 1000 may be performed by a processor of the device 120 or the user device 128.

At step 1002, a set of training data is received. The training data may comprise any combination of historical ultrasound data, augmented ultrasound data, and other metadata. For example, in some embodiments, the training data may comprise real ultrasound data received from a plurality of historical ultrasounds and augmented ultrasound data produced by augmenting the real ultrasound data to extend the data set and increase the volume of training data. Further, in some embodiments, the training data may comprise any combination of ultrasound data such as ultrasound images, ultrasound parameters, catheter selections, medical personnel data, and other data relevant to ultrasound procedures and catheter insertion procedures. In some embodiments, the set of training data may be received from one or more training data stores. For example, the set of training data may comprise a plurality of ultrasound images received from one or more medical entities and other health system data stores.

At step 1004, a machine learning model is trained based on the set of training data. For example, the machine learning model may be trained with historical ultrasound data to identify anatomical structures and other structures within subsequent real-time ultrasound data. For example, a catheter device may be identified within ultrasound images of the training data such that a similar catheter device may be identified within real-time ultrasound data by the machine learning model after training.

In some embodiments, the machine learning model may comprise a computer-vision algorithm configured to classify one or more objects within a real-time ultrasound image. Here, the computer-vision algorithm may be trained with a plurality of historical ultrasound images within the training data. For example, the computer-vision algorithm may comprise a classification model configured to classify portions of real-time ultrasound images. In some embodiments, other forms of computer vision techniques are also included. For example, any combination of image filters and other image identification techniques may be used. In some embodiments, the computer vision technique used may include any one of or combination of image segmentation, object detection, edge detection, pattern detection, image classification, and feature matching.

FIG. 11 illustrates an example of a method 1100 guiding intravenous catheter insertion relating to some embodiments of the present disclosure. Similar to as described above with respect to the method 1000, the method 1100 may be performed by at least one processor executing a set of computer-executable instructions. Further, in some embodiments, the method 1100 may be performed after the method 1000. Alternatively, or additionally, in some embodiments, the method 1100 may be performed at least in part simultaneously to the method 1000.

At step 1102, real-time ultrasound data is received. The real-time ultrasound data may be received from an ultrasound device 110 including an ultrasound probe configured to capture ultrasound images. In some embodiments, additional machine learning techniques may be applied to aid in positioning of the ultrasound device 110 and ultrasound probe to improve the image quality of the real-time ultrasound image data. In some embodiments, the real-time ultrasound data may be received from any suitable communication connection with the ultrasound device 110. For example, the ultrasound data may be transmitted via a wired or wireless connection, such as over a network or Bluetooth or other form of radio frequency connection.

In some embodiments, the real-time ultrasound data may be received prior to selection and insertion of an intravenous catheter. Alternatively, in some embodiments, the real-time ultrasound data is received after or during insertion of the intravenous catheter. Further, embodiments are contemplated in which the real-time ultrasound data may be received prior to selection and insertion of the catheter, as well as during and after insertion. For example, a first set of real-time ultrasound data may be received prior to insertion and a second set of real-time ultrasound data may be received after or during insertion.

At step 1104, one or more anatomical structures are identified within the real-time ultrasound data. In some embodiments, the one or more anatomical structures are identified using the machine learning model and/or computer vision algorithm, as described above. In some embodiments, the one or more anatomical structures identified include any one of, or combination of: a blood vessel, a vein, a nerve bundle, as well as other forms of anatomical structures present in the ultrasound image data. Additionally, or alternatively, in some embodiments, one or more other structures may be identified within the real-time ultrasound data such as, for example, a catheter device may be identified within a real-time ultrasound image.

In some embodiments, the one or more anatomical structures are identified by providing an array of pixel values from the real-time ultrasound image data to the computer vision algorithm. The computer vision algorithm analyzes the array of pixel values and identifies the one or more anatomical structures based on classification of respective portions of the array of pixel values. Similarly, a catheter or catheter needle may be identified within the real-time ultrasound data based on a portion of the array of pixel values. For example, the catheter needle may be identified based on a set of pixel values corresponding to a straight line.

Additionally, in some embodiments, the computer vision algorithm may be configured to score a plurality of potential veins or other blood vessels. Here, an insertion score may be provided for each vein identified within the real-time ultrasound data. The insertion score may be determined based on any combination of vein depth, vein diameter, blood flow rate, and other information received directly or indirectly from the real-time ultrasound data. Subsequently, the insertion scores may be compared to suggest or automatically select a particular vein for insertion. In some embodiments, a predetermined insertion score threshold is contemplated such that a potential insertion site or vein may be avoided based on receiving an insertion score below the predetermined insertion score threshold. Conversely, a particular vein or insertion site may be selected responsive to determining that the insertion score is above the predetermined insertion score threshold or above the predetermined insertion score threshold by a particular amount. Further, in some embodiments, vein suitability may be determined based at least in part on a hemodilution rate associated with the vein.

Additionally, in some embodiments, automatic error checking and other operations may be applied based on the computer vision analysis of the real-time ultrasound data. For example, a notification that the ultrasound device is faulty may be generated based on the analysis of the real-time ultrasound data responsive to detecting an error within the image data using the computer vision algorithm. In some such embodiments, the computer vision algorithm may be further trained based on historical faulty ultrasound data such that said errors may be identified. Further, one or more false artifacts may be removed or flagged within the real-time ultrasound image data. For example, common artifacts may be identified via training of the computer vision algorithm such that the artifacts may be automatically removed from the real-time image to prevent confusion for the medical personnel.

At step 1106, a blood flow is automatically estimated based on the real-time ultrasound data. For example, a blood flow of a selected vein or a potential vein considered for selection may be estimated based on one or more images from the real-time ultrasound data. In some embodiments, the blood flow may be estimated based on a vein diameter identified within the ultrasound data. For example, the vein diameter may be identified by the computer vision algorithm after the vein is identified.

At step 1108, an intravenous catheter is selected based at least in part on the real-time ultrasound data. In some embodiments, the intravenous catheter is selected from a plurality of catheters. For example, a catheter supplier or inventory data store may be accessed to review a plurality of catheters such that a suitable catheter may be selected. For example, an inventory system of catheter supplier data store may store catheter information relating to a respective plurality of catheters including catheter lengths, catheter codes/part numbers, catheter types, catheter brands, and other catheter information. In some embodiments, the catheter may be selected based on a variety of parameters such as a vein diameter of a selected vein, a selected angle of insertion, a catheter length, and other insertion parameters. A default angle of insertion of about 15 degrees may be included. However, in some embodiments, the angle of insertion may be adjusted based on one or more other insertion parameters such as a recommended hemodilution rate. Further, in some embodiments, the catheter inventory system may be automatically updated responsive to selection of a particular catheter. In some such embodiments, for example, a catheter may be automatically ordered responsive to the selection.

In some embodiments, a catheter check is contemplated. For example, a request may be generated to instruct the medical personnel to scan a universal product code associated with the catheter device. As such, a confirmation may be provided that the medical personnel has the correct catheter device prior to insertion. Alternatively, if the catheter device is incorrect, a notification may be generated for the medical personnel to locate the correct catheter device.

At step 1110, one or more suggested positioning parameters are generated for insertion of the catheter. In some embodiments, the one or more suggested positioning parameters are determined based at least in part on the real-time ultrasound data and the one or more anatomical structures identified within the real-time ultrasound data. In some embodiments, the one or more suggested positioning parameters are generated for display in real-time to a human operator such as a medical personnel performing the insertion procedure. Alternatively, or additionally, in some embodiments, the one or more suggested parameters may be provided audibly, for example, one or more verbal instructions or suggestions may be provided via one or more speakers associated with the ultrasound device 110 or the user device 128.

Alternatively, in some embodiments, the one or more suggested positioning parameters may be presented to medical personnel via an augmented reality display. For example, a medical personnel may wear a head mounted display configured to display an overlay including ultrasound data, suggestions, and other information relevant to the insertion procedure. In some embodiments, the real-time ultrasound data may be generated for display on the head mounted display overlaid with ultrasound information such as indications of identified anatomical structures, an indication of the catheter position, as well as any other measured and/or calculated parameters associated with the insertion procedure, such as, the angle of insertion, a suggested positioning change, a vein diameter, a blood flow rate, or another parameter. In some embodiments, the user interface 800A or 800B may be displayed on a display of a head mounted display device. Similarly, the indications, information, and images described above may be displayed on another form of display that is not configured to be head mounted. For example, an augmented ultrasound image may be displayed on a computer screen or a screen of a mobile device in real-time prior to and during an insertion procedure.

It should be understood that, in some embodiments, additional steps may be included in either of method 1000 or method 1100. For example, in some embodiments, an additional step for retraining the machine learning model may be included. Here, a subsequent set of training data may be received and the machine learning model may be further trained based on the subsequent set of training data. In some embodiments, the subsequent set of training data comprises ultrasound data from a plurality of catheter insertion procedures. Further still, in some embodiments, additional steps are included for storing and transmitting the real-time ultrasound data. For example, the real-time ultrasound data may be stored within an ultrasound data store for any of retraining the machine learning model, monitoring medical personnel performance, reviewing procedure parameters, and training medical personnel.

In some embodiments, a variety of filters and other augmentations of the real-time ultrasound data are contemplated. For example, in some embodiments, a three-dimensional model may be generated based on the real-time ultrasound data. In some such embodiments, the three-dimensional model provides a three-dimensional representation of the insertion region and may be provided and accessible in real-time to the medical personnel. For example, instructions and suggestions may be provided to guide a nurse or other medical personnel through the insertion procedure. Additionally, in some embodiments, the three-dimensional model may be stored within a data store such that the model may be used for subsequent monitoring or analysis of historical insertion procedures.

Further, in some embodiments, a warning or other notification may be generated and transmitted in real-time to the medical personnel, for example, to warn the medical personnel of a bad insertion site. Here, an insertion score may be generated for a plurality of respective insertion sites identified within the ultrasound data. The insertion scores may then be compared to determine an optimal or suggested insertion region along with a plurality of suggested insertion parameters for completing the insertion procedure. For example, a particular insertion site may receive a low insertion score responsive to identification of one or more nerve bundles adjacent to the insertion site. Accordingly, a warning or notification informing the medical personnel of the nerve bundles or indicating the low insertion score may be presented to the medical personnel. Said notification may be provided via any suitable form of notification described herein such as via visual display on a display device or an audible message from an audio device.

Additionally, in some embodiments, any of a plurality of instructions may be generated and provided to the medical personnel during the catheter insertion procedure. For example, in some embodiments, an instruction may be provided to the medical personnel including a suggested dwell time for the catheter device. The suggested dwell time may be selected based on any combination of the blood flow rate, the hemodilution rate, and information associated with a particular drug administered to the patient. For example, the machine learning model may automatically access a data store including pharmaceutical information relating to a particular drug being administered and determine a recommended dwell time based at least in part on the information. Alternatively, or additionally, the machine learning model may also consider patient specific factors such as a patient height, patient weight, or patient age. Further, instructions may be generated for a subsequent catheter removal procedure to guide medical personnel through removal of an inserted catheter.

Additionally, embodiments are contemplated in which ultrasound data and other patient specific insertion data may be stored, for example, to suggest a particular vein or insertion site in a subsequent procedure for the patient or a similar patient. For example, based a vein may be selected and suggested based on stored data of a successful insertion procedure. Conversely, a vein may be removed from consideration responsive to an unsuccessful insertion attempt recorded for the patient.

Although the present disclosure has been described with reference to the embodiments illustrated in the attached drawing figures, it is noted that equivalents may be employed and substitutions made herein without departing from the scope of the present disclosure as recited in the claims.

Having thus described various embodiments of the present disclosure, what is claimed as new and desired to be protected by Letters Patent includes the following:

1. A method of guiding intravenous catheter insertion into a blood vessel of a patient, the method comprising:
receiving a set of training data comprising historical ultrasound data;
training a machine learning model using the set of training data;
receiving a first set of real-time ultrasound data associated with the blood vessel from an ultrasound device prior to an insertion;
identifying a plurality of anatomical structures of the patient from the first set of real-time ultrasound data using the machine learning model;
estimating a first blood flow velocity rate associated with the blood vessel based on the first set of real-time ultrasound data;
selecting an intravenous catheter from a plurality of catheters based at least in part on the first set of real-time ultrasound data; and
generating one or more suggested positioning parameters for the intravenous catheter prior to insertion of the intravenous catheter based at least in part on the first set of real-time ultrasound data and the plurality of anatomical structures.

2. The method of claim 1, further comprising:
receiving a second set of real-time ultrasound data associated with the blood vessel from the ultrasound device after insertion of the intravenous catheter;
identifying a plurality of positioning parameters of the intravenous catheter based at least in part on the second set of real-time ultrasound data, the plurality of positioning parameters including an angle of insertion of the intravenous catheter and a depth of the intravenous catheter;
estimating a second blood flow velocity rate associated with the blood vessel based on the second set of real-time ultrasound data;
comparing the second blood flow velocity rate to the first blood flow velocity rate to determine an effect of the insertion of the intravenous catheter; and
estimating a hemodilution ratio associated with the intravenous catheter based at least in part on the second set of real-time ultrasound data, the plurality of positioning parameters, and the second blood flow velocity rate.

3. The method of claim 2, wherein the second set of real-time ultrasound data comprises:
a cross-section image of the blood vessel and the intravenous catheter; and
a side image of the blood vessel and the intravenous catheter.

4. The method of claim 1, further comprising:
automatically detecting one or more edges of the blood vessel within the first set of real-time ultrasound data.

5. The method of claim 1, wherein the machine learning model comprises a computer vision algorithm, the method further comprising:
classifying each respective structure of the plurality of anatomical structures within the first set of real-time ultrasound data using the computer vision algorithm.

6. The method of claim 5, wherein the computer vision algorithm classifies a first structure of the plurality of anatomical structures as a vein and a second structure of the plurality of anatomical structures as a nerve bundle.

7. The method of claim 6, wherein the computer vision algorithm classifies a third structure of the plurality of anatomical structures as an artery.

8. A method of guiding intravenous catheter insertion for a patient, the method comprising:
receiving a set of training data comprising historical ultrasound data;
training a machine learning model using the set of training data;
receiving real-time ultrasound data associated with the patient from an ultrasound device prior to an insertion of an intravenous catheter;
selecting the intravenous catheter from a plurality of catheters based at least in part on the real-time ultrasound data;
identifying a plurality of anatomical structures of the patient from the real-time ultrasound data using the machine learning model, the plurality of anatomical structures comprising at least one blood vessel;
estimating a first blood flow velocity rate associated with the at least one blood vessel based on the real-time ultrasound data; and
selecting an insertion site based on the first blood flow velocity rate and one or more other parameters from the real-time ultrasound data.

9. The method of claim 8, further comprising:
generating a catheter selection notification to a medical personnel, the catheter selection notification including information indicative of the intravenous catheter that is selected from the plurality of catheters.

10. The method of claim 9, further comprising:
generating a positioning notification to a medical personnel, the positioning notification including one or more guidance instructions for positioning the intravenous catheter.

11. The method of claim 8, further comprising:
generating an insertion score for the insertion site; and
comparing the insertion site to another insertion site.

12. The method of claim 8, further comprising:
storing the real-time ultrasound data in an ultrasound data store; and
retraining the machine learning model based at least in part on the real-time ultrasound data.

13. The method of claim 12, further comprising:
determining an insertion score for a medical personnel based on the real-time ultrasound data; and
storing information indicative of the insertion score.

14. The method of claim 13, further comprising:
updating training content based at least in part on the insertion score for the medical personnel.

15. A system of guiding an intravenous catheter insertion for a patient, the system comprising:
a communication connection coupled to an ultrasound device;
a notification device configured to produce one or more notifications for providing instructions to medical personnel through intravenous catheter insertion;
at least one processor; and
one or more non-transitory computer-readable media that store computer-executable instructions that, when executed by the at least one processor perform a method of guiding intravenous catheter insertion for the patient, the method comprising:
receiving a first set of real-time ultrasound data associated with the patient from the ultrasound device prior to an insertion of an intravenous catheter;
selecting the intravenous catheter from a plurality of catheters based at least in part on the first set of real-time ultrasound data;
identifying a plurality of anatomical structures of the patient from the first set of real-time ultrasound data using a machine learning model trained with a set of training data comprising a plurality of historical ultrasound data, the plurality of anatomical structures comprising at least one blood vessel;
estimating a first blood flow velocity rate associated with the at least one blood vessel based on the first set of real-time ultrasound data; and
selecting an insertion site based on the first blood flow velocity rate and one or more other parameters from the first set of real-time ultrasound data.

16. The system of claim 15, wherein the notification device comprises:
one or more displays configured to produce a visual notification for the medical personnel.

17. The system of claim 16, wherein the visual notification comprises positioning instructions for inserting the intravenous catheter including a suggested angle of insertion for the intravenous catheter.

18. The system of claim 16, wherein the notification device further comprises:
one or more speakers configured to produce an audible notification for the medical personnel, the audible notification comprising positioning instructions for inserting the intravenous catheter.

19. The system of claim 15, further comprising:
an ultrasound data store storing a plurality of historical ultrasound data.

20. The system of claim 15, wherein the method further comprises:
responsive to selecting the intravenous catheter from the plurality of catheters, automatically updating an inventory system associated with the plurality of catheters.
